(19) United States
(12) Patent Application Publication
(10) Pub. No.: US 2026/0240128 A1
(43) Pub. Date: Aug. 20, 2026

(54) SYSTEMS AND METHODS FOR RAISING SUPERWORMS AND OTHER INSECTS

(71) Applicant: Fluker's Cricket Farm, Inc., Port Allen, LA (US)

(72) Inventors: Brendon Michael Sassmannshaus, LaBelle, FL (US); Trina Lynn Chiasson, LaBelle, FL (US); Scott Ungar Campbell, Dartmouth (CA)

(73) Assignee: Fluker's Cricket Farm, Inc., Port Allen, LA (US)

(21) Appl. No.: 19/480,408

(22) PCT Filed: May 1, 2024
(86) PCT No.: PCT/US2024/027144

§ 371 Date: Oct. 31, 2025

Related U.S. Application Data
(60) Provisional application No. 63/499,670, filed on May 2, 2023.

(51) Int. Cl. A01K 67/362

(52) U.S. Cl. CPC A01K 67/362

(57) ABSTRACT

The present disclosure generally relates to systems and methods for raising superworms and other insects. Certain aspects are generally directed to articles having a plurality of cavities that superworms or other larvae can enter and then pupate. For instance, the article may have tens, hundreds, or thousands of such cavities. It has been found that superworms prefer cavities of certain dimensions, while cavities that are too large or too small are not as conducive to pupation by superworms. In some cases, the superworms will also move about the article to look for an unoccupied cavity in which to enter, which can be used to facilitate loading of the article with the superworms, for example, since an article can be exposed to a colony of superworms, which then may enter the cavities themselves. In addition, in some embodiments, after pupation, the humidity within the cavities can be controlled, e.g., by using a mesh or through-holes to regulate airflow. Other aspects are generally directed to systems of making or using such articles, kits involving such articles, or the like.

### RELATED APPLICATIONS

This application claims the benefit of U.S. Provisional Patent Application Ser. No. 63/499,670, filed May 2, 2023, entitled “Systems and Methods for Raising Superworms and Other Insects,” incorporated herein by reference in its entirety.

### FIELD

The present disclosure generally relates to systems and methods for raising superworms and other insects.

### BACKGROUND

Superworms are the larval form of certain darkling beetles (e.g., Zophobas morio). Superworms have been raised as food, for example, for the pet industry, or as a source of protein for various applications. Superworms are relatively easy to grow, and can be fed a variety of sources of food, such as grains, fruit, vegetables, decomposing organic matter, or even certain types of plastics such as polystyrene. In order to maintain a colony of superworms, some of the superworms must be allowed to pupate to mature into adults. A female darkling beetle can lay over 2,000 eggs to produce the next generation of superworms. However, it can be difficult to cause the superworms to pupate. If the superworms are kept together in a container, they will not pupate. Instead, the superworms must be isolated from each other for several days. Such isolation causes the superworms to pupate, and eventually become adults. While relatively small populations of superworms can be isolated individually (for example, by moving individual superworms into film canisters or small deli containers by hand), this quickly becomes impractical when scaled up to hundreds or thousands of superworms. Accordingly, more advanced techniques to isolate superworms are needed.

### SUMMARY

The present disclosure generally relates to systems and methods for raising superworms and other insects. The subject matter of the present disclosure involves, in some cases, interrelated products, alternative solutions to a particular problem, and/or a plurality of different uses of one or more systems and/or articles.

One aspect is generally directed to a device. In one set of embodiments, the device comprises an article defining a plurality of cavities each having an opening. In some cases, at least 50% of the cavities in the article have a cross-sectional dimension of between 10 mm and 14 mm and a depth of between 60 mm and 90 mm. In certain embodiments, at least 5% or at least 50% of the cavities contain a superworm or other insect larvae.

The device, in another set of embodiments, comprises an article defining a plurality of cavities each having an opening. In some cases, at least 50% of the cavities in the article have a cross-sectional dimension of between 10 mm and 14 mm and a depth of between 60 mm and 90 mm. In certain instances, a surface opposed to the cavity openings defines a plurality of through-holes.

In yet another set of embodiments, the device comprises an article that comprises a plurality of cavities. In some embodiments, at least 50% of the cavities in the article have a cross-sectional dimension of between 10 mm and 14 mm and a depth of between 60 mm and 90 mm. In certain cases, a surface opposed to the cavity openings has a void area of at least 50%.

In still another set of embodiments, the device comprises an article defining a plurality of cavities. In some cases, at least 50% of the cavities in the article have a cross-sectional dimension of between 10 mm and 14 mm and a depth of between 60 mm and 90 mm, and a surface opposed to the cavity openings permits air flow into the cavity.

The device, in yet another set of embodiments, comprises an article defining a plurality of cavities. In certain cases, at least 50% of the cavities in the article have a cross-sectional dimension of between 10 mm and 14 mm and a depth of between 60 mm and 90 mm, and a moisture-absorbing surface opposed to the cavity openings of the plurality of cavities.

According to another set of embodiments, the device comprises an article defining a plurality of cavities each having an opening having an cross-sectional opening dimension. In some embodiments, at least 5%, 50%, or more of the cavities contain an insect larvae.

The device, in yet another set of embodiments, comprises an article defining a plurality of cavities each having an opening. In some cases, at least 50% of the cavities in the article may have a cross-sectional dimension of between 10 mm and 14 mm and a depth of between 60 mm and 90 mm.

In one set of embodiments, the device comprises an article defining a plurality of cavities each having an opening, at least 5% of the cavities in the article having a cross-sectional dimension of between 10 mm and 14 mm and a depth of between 60 mm and 90 mm. In some cases, at least 5% of the cavities contain an insect larvae.

In another set of embodiments, the device comprises an article defining at least 10 cavities each having an opening, a cross-sectional dimension of between 10 mm and 14 mm, and a depth of between 60 mm and 90 mm. In certain embodiments, at least 5% of the cavities contain a superworm.

The device, in yet another set of embodiments, comprises an article defining at least 10 cavities each having an opening, a cross-sectional dimension of between 10 mm and 14 mm, and a depth of between 60 mm and 90 mm. In some embodiments, a surface opposed to the cavity openings defines a plurality of through-holes.

The device, in still another set of embodiments, comprises an article defining at least 10 cavities each having an opening, a cross-sectional dimension of between 10 mm and 14 mm, and a depth of between 60 mm and 90 mm. In certain embodiments, a surface opposed to the cavity openings has a void area of at least 50%.

According to yet another set of embodiments, the device comprises an article defining at least 10 cavities, each having an opening, a cross-sectional dimension of between 10 mm and 14 mm, a depth of between 60 mm and 90 mm, and a surface opposed to the cavity openings that permits air flow into the cavity.

The device, in still another set of embodiments, comprises an article defining at least 10 cavities, each having a cross-sectional dimension of between 10 mm and 14 mm, a depth of between 60 mm and 90 mm, and a moisture-absorbing surface opposed to the cavity openings of the plurality of cavities.

In one set of embodiments, the device comprises an article defining at least 10 cavities, each having an opening, a cross-sectional dimension of between 10 mm and 14 mm, and a depth of between 60 mm and 90 mm.

The device, according to another set of embodiments, comprises an article defining at least 10 cavities each having an opening, a cross-sectional dimension of between 10 mm and 14 mm, and a depth of between 60 mm and 90 mm. In some embodiments, at least 5% of the cavities contain an insect larvae.

Another aspect is generally drawn to a method. In accordance with one set of embodiments, the method comprises exposing an article defining a plurality of cavities each having an opening, at least 50% of the cavities in the article having a cross-sectional dimension of between 10 mm and 14 mm and a depth of between 60 mm and 90 mm, to a colony of at least 100 superworms or other insect larvae.

In another set of embodiments, the method comprises exposing an article defining a plurality of cavities each having an opening to a colony of at least 100 superworms or other insect larvae for a period of time such that the superworms or other insect larvae move into at least 5% or at least 50% of the cavities in the article.

The method, in still another set of embodiments, comprises exposing an article defining a plurality of cavities each having an opening to a colony of at least 100 superworms or other insect larvae, and allowing the superworms or other insect larvae to search the article for empty cavities.

The method, in accordance with yet another set of embodiments, comprises flowing air through a plurality of cavities defined within an article. In some embodiments, at least 5% or at least 50% of the cavities contains a superworm or other insect larvae.

In addition, the method, in another set of embodiments, comprises providing an article defining a plurality of cavities, and using a robot to place superworms or other insect larvae within the cavities.

In one set of embodiments, the method comprises exposing an article defining a plurality of cavities each having an opening, at least 5% of the cavities in the article having a cross-sectional dimension of between 10 mm and 14 mm and a depth of between 60 mm and 90 mm, to a colony of at least 100 insect larvae.

The method, in accordance with another set of embodiments, comprises exposing an article defining a plurality of cavities each having an opening to a colony of at least 100 insect larvae for a period of time such that the insect larvae move into at least 5% of the cavities in the article.

In still another set of embodiments, the method comprises exposing an article defining a plurality of cavities each having an opening to a colony of at least 100 insect larvae, and allowing the insect larvae to search the article for empty cavities.

Yet another set of embodiments is directed to a method comprising exposing an article defining at least 10 cavities each having an opening, a cross-sectional dimension of between 10 mm and 14 mm, and a depth of between 60 mm and 90 mm to a colony of at least 100 insect larvae.

The method, in another set of embodiments, comprises flowing air through a plurality of cavities defined within an article, wherein at least 5% of the cavities contains an insect larvae.

In still another set of embodiments, the method comprises providing an article defining a plurality of cavities, and using a robot to place insect larvae within the cavities.

In yet another set of embodiments, the method comprises exposing an article defining at least 10 cavities each having an opening, a cross-sectional dimension of between 10 mm and 14 mm, and a depth of between 60 mm and 90 mm to a colony of at least 100 superworms. In another aspect, the present disclosure encompasses methods of making one or more of the embodiments described herein, for example, an article for containing superworms or other insect larvae. In still another aspect, the present disclosure encompasses methods of using one or more of the embodiments described herein, for example, an article for containing superworms or other insect larvae.

Other advantages and novel features of the present disclosure will become apparent from the following detailed description of various non-limiting embodiments of the disclosure when considered in conjunction with the accompanying figures.

### BRIEF DESCRIPTION OF THE DRAWINGS

Non-limiting embodiments of the present disclosure will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

FIG. 1 illustrates an article in accordance with one embodiment of the invention; and

FIG. 2 is a photograph illustrating superworms within the cavities in an article, in yet another embodiment.

### DETAILED DESCRIPTION

The present disclosure generally relates to systems and methods for raising superworms and other insects. Certain aspects are generally directed to articles having a plurality of cavities that superworms or other larvae can enter and then pupate. For instance, the article may have tens, hundreds, or thousands of such cavities. It has been found that superworms prefer cavities of certain dimensions, while cavities that are too large or too small are not as conducive to pupation by superworms. In some cases, the superworms will also move about the article to look for an unoccupied cavity in which to enter, which can be used to facilitate loading of the article with the superworms, for example, since an article can be exposed to a colony of superworms, which then may enter the cavities themselves. In addition, in some embodiments, after pupation, the humidity within the cavities can be controlled, e.g., by using a mesh or through-holes to regulate airflow. Other aspects are generally directed to systems of making or using such articles, kits involving such articles, or the like.

As mentioned, superworms are the larval form of darkling beetles. There are a variety of different species of darkling beetles, including but not limited to Zophobas morio. Indeed, many darkling beetle species are members of the Tenebrionidae beetle family, of which over 20,000 such species have been identified. The beetles hatch from eggs as larvae (colloquially termed “worms” or “superworms”), which transform into pupae, then mature as beetles who are sexually mature and lay eggs, thereby completing the lifecycle. However, in order to raise superworms, a population of the superworms must be allowed to pupate to become sexually mature adult beetles capable of laying eggs to produce the next generation. It should be understood that the term “superworms,” as used herein, does not refer to only Zophobas morio, but to other species as well, including other members of the Tenebrionidae beetle family.

When kept together, superworm larvae will often not pupate, but will instead remain in the larval stage indefinitely. Accordingly, the superworms need to be isolated from each other in order to allow pupation to occur. Without wishing to be bound by any theory, it is believed that in nature, superworms will instinctively look for a hole in which to pupate without being disturbed. If a superworm starts to pupate in a hole and is disturbed, the superworm may abort pupation to search for another hole.

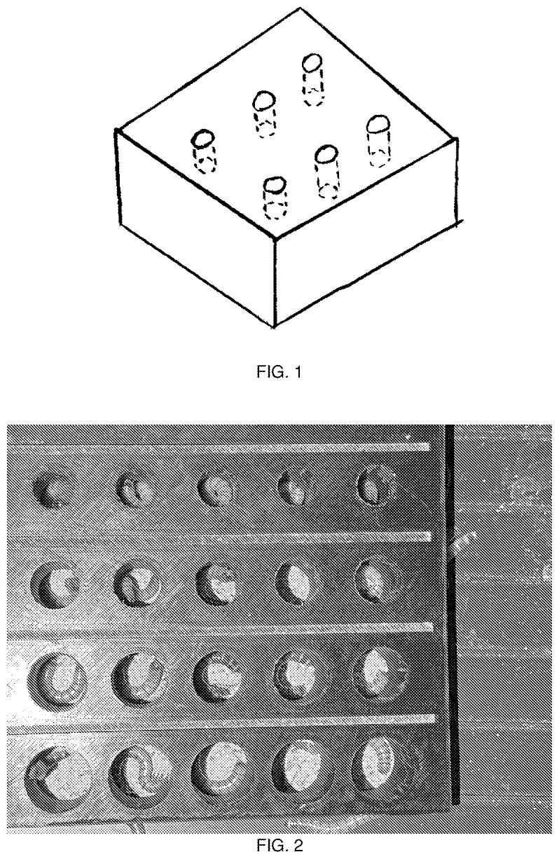

Accordingly, certain aspects as discussed herein are directed to articles that can be used by superworms for pupation, and techniques for making and using such articles. One such non-limiting example is now described with reference to FIG. 1. In this figure, article is shown, defining a plurality of cavities exposed to a surface of the article. The cavities may independently be of the same or different sizes. For example, as shown here, all of the cavities are of the same size. In addition, some or all the cavities may be partial, and/or some or all of the cavities may be through-holes that go through the article. The article may have a square or rectangular shape, although other shapes are also possible in other embodiments. For instance, in FIG. 1, the article has a substantially rectangular shape.

The cavities may have a shape that a superworm (or other insect) finds appealing. As mentioned, and without wishing to be bound by any theory, it is believed that superworms are attracted to cavities of a certain size in which to pupate. Superworms typically pupate in a setting in which they believe to be substantially isolated from other superworms. Accordingly, superworms may be attracted to cavities they believe are spacious enough to pupate in, but not so spacious that other superworms may wish to enter. Various examples of suitable dimensions for the cavities are discussed in detail herein.

In some cases, it is believed that under the right conditions, superworms may independently move about a surface to search for a cavity upon which it can enter in order to pupate. If a superworm finds a cavity already containing another superworm, the superworm may continue its search rather than enter that cavity. In this way, a plurality of superworms may crawl about the surface of an article until they have distributed themselves in the cavities such that substantially each of the cavities contains a single superworm. Although the superworms may also be distributed using other techniques (e.g., by hand, or by using a robot such as discussed herein, etc.), in some embodiments, no distribution technique is required, as the superworms will independently move and distribute themselves accordingly within the cavities, e.g., such that substantially each of the cavities contains a single superworm.

The superworms, after selecting a suitable cavity, may pupate if left relatively undisturbed, e.g., isolated from other superworms. After about 7-10 days, the superworms will form pupae (and after about 1-2 weeks, the pupae will mature into adult beetles), although in some cases, the superworms may take longer to from pupae (e.g., 2 or 3 weeks). In some embodiments, the humidity of the cavities may be controlled, e.g., to prevent too much moisture from being contained within the cavities. Without wishing to be bound by any theory, it is believed that high moisture may encourage fungi or molds to grow on the pupae, which are detrimental to the pupae. Accordingly, in some embodiments, the cavities may include through-holes to allow air flow into the cavity, moisture-absorbing surfaces, or the like.

The above discussion is a non-limiting example of one embodiment of the present invention that is generally directed to articles containing cavities for superworms or other insects. However, other embodiments are also possible. Accordingly, more generally, various aspects of the invention are directed to various systems and methods for systems and methods for raising superworms and other insects.

For example, while the above discussion was presented with respect to superworms (e.g., the larval phase of Zophobas morio, or other species), it should be understood that the present disclosure is not so limited in other aspects, and that in a variety of other embodiments, other insect larvae, or other organisms, may be present, e.g., instead of and/or in addition to superworms. For example, other insect larvae that can be raised using articles and methods such as those described herein include mealworms, hornworms, waxworms, Tenebrio molitor, Tribolium castaneeum, Alphitobius diaperinus (lesser mealworm), Tribolium confusum, Tribolium destructor, Gnathocerus cornutus, Eleodes obscurus, Ulomoides dermestoides, Dalotia coriaria, or the like. For example, in some embodiments, an article such as described herein may be exposed to insect larvae such as these, or colonies of larvae that may include one or more of these, and/or other types of insect larvae, etc.

In one aspect, superworms and/or other insect larvae may be housed in an article defining one or more cavities therein. The article may be formed out of a variety of materials, and may include one or several materials. For example, the article may be formed out of plastic (e.g., acrylic or polymethylmethacrylate, polycarbonate, HDPE, polypropylene, polyethylene terephthalate, polyvinylchloride, acrylonitrile-butadiene-styrene, acrylic, etc.), delrin, wood, metal, cardboard, paper, stainless steel, or the like. In some cases, wood may be useful due to its moisture-absorbent properties, e.g., as discussed herein. In addition, in certain embodiments, at least 50 w% of the article comprises a single material (e.g., a plastic or a wood, etc.), and in some cases, at least 60 wt %, at least 70 wt %, at least 80 wt %, or at least 90 wt % of the article may comprise a single material.

The article may define one or more cavities, which may be present on any surface of the article, in one set of embodiments. There may be any number of cavities present. For example, there may be at least 5, at least 10, at least 20, at least 30, at least 50, at least 100, at least 30, at least 500, at least 1,000, at least 3,000, at least 5,000, or at least 10,000 cavities within an article. The cavities may be present in any suitable configuration. For example, the cavities may be arranged in a rectangular arrangement, a hexagonal arrangement, a circular arrangement, an irregular arrangement, or the like.

In some embodiments, all of the cavities may open to the same side of the material. A non-limiting example is shown in FIG. 1. However, in other embodiments, the cavities may open to different surfaces of the article. As a non-limiting example, about 50% of the cavities may open to a first surface of the material and about 50% may open to a second surface of the material.

The cavities may have any suitable size or shape. For example, at least 1%, at least 5%, at least 10%, at least 20%, at least 30%, at least 40%, at least 50%, at least 60%, at least 70%, at least 80%, at least 90%, or substantially all of the cavities may be cylindrical. As another example, at least 1%, at least 5%, at least 10%, at least 20%, at least 30%, at least 40%, at least 50%, at least 60%, at least 70%, at least 80%, at least 90%, or substantially all of the cavities may have an opening that is circular, square, triangular, hexagonal, irregular, or other suitable shapes.

In one embodiment, a cavity may have an opening having an average cross-sectional dimension of at least 5 mm, at least 8 mm, at least 9 mm, at least 10 mm, at least 11 mm, at least 12 mm, at least 13 mm, at least 14 mm, at least 15 mm, etc. In some cases, a cavity may have an opening having an average cross-sectional dimension that is no greater than 20 mm, no greater than 18 mm, no greater than 17 mm, no greater than 16 mm, no greater than 15 mm, no greater than 14 mm, no greater than 13 mm, no greater than 12 mm no greater than 11 mm, no greater than 10 mm, etc. Combinations of any of these dimensions are also possible in certain embodiments. For instance, a cavity may have an average cross-sectional dimension that is between 10 mm and 14 mm, between 5 mm and 20 mm, between 5 mm and 14 mm, between 10 mm and 20 mm, between 9 mm and 15 mm, between 8 mm and 16 mm, between 10 mm and 15 mm, or the like. If the opening is non-circular, then the average cross-sectional dimension may be taken as the diameter of a perfect circle having the same area as the opening of the cavity.

The cavity may also have any suitable depth. For instance, the cavity may have an average depth of at least 40 mm, at least 50 mm, at least 55 mm, at least 60 mm, at least 65 mm, at least 70 mm, at least 75 mm, at least 80 mm, at least 85 mm, at least 90 mm, etc. In some cases, a cavity may have an average depth of no more than 110 mm, no more than 100 mm, no more than 95 mm, no more than 90 mm, no more than 85 mm, no more than 80 mm, no more than 75 mm, no more than 70 mm, no more than 65 mm, no more than 60 mm, etc. In addition, combinations of any of these depths are also possible in certain cases. For instance the depth of a cavity may be between 60 mm and 90 mm, between 55 mm and 15 mm, between 50 mm and 100 mm, between 65 mm and 85 mm, between 60 mm and 100 mm, between 80 mm and 100 mm, etc.

A cavity may also have any suitable volume in another embodiment. For example, a cavity may have a volume of at least 3cm 3 , at least 4cm 3 , at least 5cm 3 , at least 6cm 3 , at least 7cm³ , at least 8cm³ , at least 9cm³ , at least 10cm³ , at least 11cm³ , at least 12cm³ , at least 13cm³ , at least 14cm³ , at least 15cm³ , etc. In addition, a cavity may have a volume of no more than 15cm 3 , no more than 14cm 3 , no more than 13cm 3 , no more than 12cm 3 , no more than 11cm³ , no more than 10cm³ , no more than 9cm³ , no more than 8cm³ , no more than 7cm³ , no more than 6cm³ , no more than 5cm³ , no more than 4cm³ , no more than 3 cm³, etc. Combinations of any of these are also possible; for instance, a cavity may have a volume of between 4 cm³ and 14cm³ , between 5 cm³ and 15cm³ , between 3 cm³ and 10cm³ , between 10 cm³ and 15cm³ , between 8 cm³ and 12cm³ , or the like.

As mentioned, the cavities may each independently have the same or different shapes, sizes, or dimensions, including any of the dimensions provided herein. For example, at least 1%, at least 5%, at least 10%, at least 20%, at least 30%, at least 40%, at least 50%, at least 60%, at least 70%, at least 80%, at least 90%, or substantially all of the cavities may be substantially identical. However, in some cases, different size cavities may be present, e.g., as is shown in FIG. 2.

In one set of embodiments, as mentioned, the cavities may have a shape that a superworm (or other insect) finds appealing. For example, the cavities may be of a shape or size that allows only a single superworm or other insect to fit (e.g., to pupate), or the cavities may be of a shape or size that, while it can physically contain more than one superworm (or other insect), is suitable sized that only a single superworm (or other insect) will enter and pupate, e.g., other superworms (or other insects) will not willingly enter to pupate, and if more than superworm (or other insect) is present, the other superworms (or other insects) may leave until only a single superworm (or other insect) is left behind. Unexpectedly, it has been found that there is a relatively narrow range of sizes and/or dimensions that superworms will find appealing. Cavities that are too big may discourage superworms from pupating therein, e.g., due to their perceived risk of exposure to predators from outside, or to the undesirability of having another superworm enter, etc. However, cavities that are too small may also discourage superworms from pupating therein, e.g., because the superworm cannot physically enter (due to the size of the cavity), or because the superworm is too constrained within the cavity from feeling comfortable in pupating. Accordingly, superworms may surprisingly prefer cavities of only a certain size for pupating. For example, such a cavity may have a cross-sectional dimension of between 10 mm and 14 mm and a depth of between 60 mm and 90 mm, or other dimensions such as those described herein.

In some aspects, some or all of the cavities within an article may be constructed and arranged such that the humidity within those cavities may be controlled. As discussed, without wishing to be bound by any theory, it is believed that too much humidity or moisture present within a cavity may encourage fungi or molds to grow on the pupae, which are detrimental to the pupae. Accordingly, in some embodiments, at least some of the cavities may be constructed and arranged to allow the humidity therein to be controlled. For instance, in some cases, at least some of the cavities may contain one or more through-holes to allow air flow into the cavity. For instance, a surface of the cavity may have a void area of at least 10% or more, e.g., to permit air flow through at least a portion of the cavity to occur. As another example, at least some of the cavities may contain one or more moisture-absorbing surfaces or other materials.

For example, in one set of embodiments, one or more of the cavities may contain one or more through-holes, e.g., in a surface defining the cavity, to permit air flow into or through the cavity. The surface may be any suitable surface, e.g., a surface that is opposed to the opening of the cavity. There may be any suitable number of through-holes in the surface, e.g., 1, 2, 3, 4, or more. For instance, there may be at least 5, at least 10, at least 15, at least 20, at least 25, at least 30, etc. through-holes defined in a surface of the cavity. The holes may independently be of the same or different sizes.

In certain cases, some or all the through-holes may have a shape, size, or cross-sectional dimension that does not allow a superworm (or other insect) to be able to crawl through. For example, the through-holes may have an average dimension of less than 10 mm, less than 9 mm, less than 8 mm, less than 7 mm, less than 6 mm, less than 5 mm, less than 4 mm, less than 3 mm, less than 2 mm, less than 1 mm, etc. In addition, in some embodiments, the through-holes may exit to a region external of the article, for instance, the through-holes may be present in a surface that is an outside surface of the article. In some cases, the article may comprise a wire mesh surface that is positioned opposite of the openings of the cavities. In some cases, the through-holes may be present such that the void area of the surface is, for example, at least 1%, at least 5%, at least 10%, at least 20%, at least 30%, at least 40%, at least 50%, at least 60%, at least 70%, at least 80%, at least 85%, at least 90%, or at least 95% of the surface. As an illustrative non-limiting example, the surface may be formed of a mesh of wires, and most of the surface is defined by the empty spaces between the wires, e.g., defining a series of through-holes.

However, it should be understood that techniques other than surfaces with through-holes can also be used to allow the humidity within a cavity therein to be controlled. For instance, in one set of embodiments, a moisture-absorbing surface may be present, e.g., defining a wall of the cavity, and/or being located internally of the cavity. For example, in some embodiments, a surface of the cavity may be formed out of a moisture-absorbing material.

Non-limiting examples of moisture-absorbing materials include wood (e.g., balsa, pine, beech, redwood, oak, willow, ash, walnut, butternut, etc.), desiccant (e.g., alumina, bentonite clay, calcium chloride, calcium oxide, calcium sulfate, cobalt (II) chloride, copper (I) sulfate, lithium chloride, lithium bromide, magnesium chloride hexahydrate, magnesium sulfate, magnesium perchlorate, phosphorus pentoxide, potassium carbonate, potassium hydroxide, rice, silica gel, sodium chlorate, sodium chloride, sodium hydroxide, sodium sulfate, sucrose, etc.), clays (e.g., bentonite), fabrics or fibers (e.g., cotton, hemp, bamboo, linen, rayon, wool, fleece, flannel, microfiber, etc.) or the like. For example, a surface defining at least a portion of the cavity, such as a surface that is opposed to the opening of the cavity, may be formed of wood, or other moisture-absorbing materials such as those described herein. Certain types of wood may be particularly useful in some embodiments, e.g., due to their ability to absorb moisture.

In one aspect, the superworms (or other insects) may be added to the cavities using a suitable robot. For example, the superworms (or other insects) may be taken from a colony of superworms (or other insects), and individually positioned into the cavities of the article such that the superworms (or other insects) are present in at least 1%, at least 5%, at least 10%, at least 20%, at least 30%, at least 40%, at least 50%, at least 60%, at least 70%, at least 80%, at least 90%, or at least 95% of the cavities within the article. In some cases, the robot may position the superworms (or other insects) in the cavities such that at least 1%, at least 5%, at least 10%, at least 20%, at least 30%, at least 40%, at least 50%, at least 60%, at least 70%, at least 80%, at least 90%, or at least 95% of the cavities within the article that contains a superworm (or other insects) contains only one superworm (or other insect). In some embodiments, a variety of robots may be used, many of which can be readily obtained commercially. For instance, in one set of embodiments, the robot may comprise an articulated arm that is able to grasp or contain a superworm or other insect (e.g., from a colony of superworms or other insects) and move it into a cavity, e.g., in an article such as is described herein. As another non-limiting example, the robot may include a controlled slide that allows superworms (or other insects) to “slide” into a plurality of cavities in an article, e.g., as a way to rapidly load the cavities with superworms (or other insects).

In addition, as mentioned, certain aspects are directed to the observation that certain superworms prefer cavities of certain dimensions, while cavities that are too large or too small are not as conducive to pupation by superworms. Without wishing to be bound by any theory, it is believed that superworms will instinctively look for a hole, e.g., in a tree or a nook in a cave, in which the superworm will be able to pupate without being disturbed. Accordingly, in some embodiments, an article such as is discussed herein may be exposed to one or more superworms (for example, a colony of superworms), and the superworms may be allowed to explore the article, e.g., in order to look for a suitable empty cavity (e.g., not already containing a superworm) in which it can enter and pupate.

Thus, in some cases, an article such as described herein may be exposed to a colony of superworms (or other insects) to allow the superworms (or other insects) to search the article in order to look for empty cavities. During such exposure over a suitable period of time, the superworms (or other insects) may spontaneously crawl around the article, and thus explore the cavities within the article. For instance, the article may be exposed to the superworms (or other insects) for at least 3 hours, at least 6 hours, at least 12 hours, at least 18 hours, at least 24 hours, at least 36 hours, at least 48 hours, etc., and/or for no more than 72 hours, no more than 48 hours, no more than 36 hours, no more than 24 hours, no more than 18 hours, no more than 12 hours, no more than 3 hours etc. In some cases, a precise time of exposure may not be critical, e.g., an article may be exposed to a colony of superworms (or other insects) for about a day, for between 12 hours and 24 hours, between 6 hours and 18 hours, between 18 hours and 36 hours, etc., or any other suitable combination of times. However, in other embodiments, longer or shorter exposure periods can also be used. In some cases, the article may be exposed to superworms (or other insects) such that the superworms (or other insects) move into and/or are present in at least 1%, at least 5%, at least 10%, at least 20%, at least 30%, at least 40%, at least 50%, at least 60%, at least 70%, at least 80%, at least 90%, or at least 95% of the cavities within the article. In some cases, the superworms (or other insects) may move into all of the cavities within the article. As mentioned, the superworms (or other insects) may move into the article such that some or all of the cavities contains superworms (or other insects), and in such cases, such that at least 1%, at least 5%, at least 10%, at least 20%, at least 30%, at least 40%, at least 50%, at least 60%, at least 70%, at least 80%, at least 90%, or at least 95% of the cavities within the article that contains a superworm (or other insects) contains only one superworm (or other insect).

The colony of superworms (or other insects) may have any suitable number of superworms (or other insects) present. In some cases, the colony of superworms (or other insects) contains so many superworms (or other insects) that accurate counting of the superworms (or other insects) is difficult or impossible. Thus, for example, a colony may contain at least 5, at least 10, at least 30, at least 50, at least 100, at least 300, at least 500, at least 1,000, at least 3,000, at least 5,000, at least 10,000, at least 30,000, at least 50,000, or at least 100,000 superworms (or other insects). In some cases, the colony of superworms (or other insects) may be contained in a suitable container (e.g., a bin), and the article placed within the container such that the superworms (or other insects) are able to crawl about the article and search for empty cavities. This may represent a significant improvement over manual techniques where a human individually positions, by hand, hundreds or thousands of superworms (or other insects) within a plurality of containers such that each container contains only one superworm (or other insects).

After the superworms (or other insects) are in the cavities, the article may be removed from the colony of superworms (or other insects) in accordance with certain aspects, and left in an undisturbed location to allow the superworms (or other insects) to pupate and mature into adult beetles, as discussed. For instance, after about 7-10 days, the superworms (or other insects) may form pupae, and after about 1-2 weeks, the pupae will mature into adult beetles (or other insects). In addition, in some embodiments, the article may also be moved during the pupation process. For instance, the article may be rotated or turned upside down, for example, after about halfway through the pupation process.

U.S. Provisional Patent Application Ser. No. 63/499,670, filed May 2, 2023, entitled “Systems and Methods for Raising Superworms and Other Insects,” is incorporated herein by reference in its entirety.

The following examples are intended to illustrate certain embodiments of the present disclosure, but do not exemplify the full scope of the disclosure.

### EXAMPLE 1

In this example, an article defining a plurality of cavities was prepared, and exposed to a bin containing hundreds of superworms. The article was fabricated from plastic, and the cavities were cylindrical, roughly 12 mm wide with a depth of about 3 inches (about 76 mm). The cavities were arranged in a generally hexagonal pattern.

The article was placed in a container containing several thousand superworms. The article was left undisturbed within the container. Over a period of time of about a day, superworms began crawling around the surface of the article, and occasionally entering cavities that they found empty. Afterwards, the article was removed, and it was found that at many of the cavities now each contained a single superworm.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the word “about” is used herein in reference to a number, it should be understood that still another embodiment of the disclosure includes that number not modified by the presence of the word “about.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method, comprising:
exposing an article defining a plurality of cavities each having an opening, at least 5% of the cavities in the article having a cross-sectional dimension of between 10 mm and 14 mm and a depth of between 60 mm and 90 mm, to a colony of at least 100 superworms.

2. The method of claim 1, comprising exposing the article to the colony for a period of time such that the superworms move into at least 10% of the cavities in the article.

3. The method of claim 2, wherein the superworms move into at least 10% of the cavities in the article such that at least 10% of the cavities contains only one superworm.

4. The method of any one of claims 1-3, further comprising allowing the superworms to search the article for empty cavities.

5. The method of any one of claims 1-4, further comprising using a robot to place superworms within the cavities.

6. The method of any one of claims 1-5, wherein at least 90 wt % of the article comprises plastic.

7. The method of any one of claims 1-6, wherein at least 90 wt % of the article comprises wood.

8. The method of any one of claims 1-7, wherein at least 50% of the cavities are cylindrical.

9. The method of any one of claims 1-8, wherein at least 50% of the cavities have substantially identical dimensions.

10. The method of any one of claims 1-9, wherein the article comprises at least 100 cavities.

11. The method of any one of claims 1-10, wherein the article comprises at least 1,000 cavities.

12. The method of any one of claims 1-11, wherein at least 90% the cavities in the article have a cross-sectional dimension of between 10 mm and 14 mm and a depth of between 60 mm and 90 mm.

13. The method of any one of claims 1-12, wherein the plurality of cavities are arranged in a hexagonal arrangement.

14. The method of any one of claims 1-13, wherein the plurality of cavities are arranged in a rectangular arrangement.

15. The method of any one of claims 1-14, wherein the colony is a colony of at least 1,000 superworms.

16. The method of any one of claims 1-15, wherein the colony is a colony of at least 10,000 superworms.

17. The method of any one of claims 1-16, wherein a surface opposed to the cavity openings defines a plurality of through-holes.

18. The method of any one of claims 1-17, wherein a surface opposed to the cavity openings has a void area of at least 50%.

19. The method of any one of claims 1-18, wherein a surface opposed to the cavity openings permits air flow into the cavities.

20. The method of any one of claims 1-19, wherein a surface opposed to the cavity openings is moisture-absorbing.

21. The method of any one of claims 1-20, further comprising flowing air through the plurality of cavities.

22. A method, comprising:
exposing an article defining a plurality of cavities each having an opening to a colony of at least 100 superworms for a period of time such that the superworms move into at least 5% of the cavities in the article.

23. The method of claim 22, wherein at least 50% of the cavities in the article having a cross-sectional dimension of between 10 mm and 14 mm and a depth of between 60 mm and 90 mm.

24. The method of any one of claims 22 or 23, comprising allowing the superworms to search the article for empty cavities.

25. The method of any one of claims 22-24, wherein the superworms move into at least 50% of the cavities in the article such that at least 50% of the cavities contains only one superworm.

26. The method of any one of claims 22-25, wherein at least 90 wt % of the article comprises plastic.

27. The method of any one of claims 22-26, wherein at least 90 wt % of the article comprises wood.

28. The method of any one of claims 22-27, wherein at least 50% of the cavities are cylindrical.

29. The method of any one of claims 22-28, wherein at least 50% of the cavities have substantially identical dimensions.

30. The method of any one of claims 22-29, wherein the article comprises at least 100 cavities.

31. The method of any one of claims 22-30, wherein at least 90% the cavities in the article have a cross-sectional dimension of between 10 mm and 14 mm and a depth of between 60 mm and 90 mm.

32. The method of any one of claims 22-31, wherein a surface opposed to the cavity openings defines a plurality of through-holes.

33. The method of any one of claims 22-32, wherein a surface opposed to the cavity openings permits air flow into the cavities.

34. The method of any one of claims 22-33, wherein a surface opposed to the cavity openings is moisture-absorbing.

35. A method, comprising:
exposing an article defining a plurality of cavities each having an opening to a colony of at least 100 superworms; and
allowing the superworms to search the article for empty cavities.

36. The method of claim 35, wherein at least 50% of the cavities in the article having a cross-sectional dimension of between 10 mm and 14 mm and a depth of between 60 mm and 90 mm.

37. The method of any one of claims 35-36, comprising allowing the superworms to search the article for empty cavities such that the superworms move into at least 50% of the cavities in the article.

38. The method of claim 37, wherein the superworms move into at least 50% of the cavities in the article such that at least 50% of the cavities contains only one superworm.

39. The method of any one of claims 35-38, wherein at least 90 wt % of the article comprises plastic.

40. The method of any one of claims 35-39, wherein at least 90 wt % of the article comprises wood.

41. The method of any one of claims 35-40, wherein at least 50% of the cavities are cylindrical.

42. The method of any one of claims 35-41, wherein at least 50% of the cavities have substantially identical dimensions.

43. The method of any one of claims 35-42, wherein the article comprises at least 100 cavities.

44. The method of any one of claims 35-43, wherein at least 90% the cavities in the article have a cross-sectional dimension of between 10 mm and 14 mm and a depth of between 60 mm and 90 mm.

45. The method of any one of claims 35-44, wherein a surface opposed to the cavity openings defines a plurality of through-holes.

46. The method of any one of claims 35-45, wherein a surface opposed to the cavity openings permits air flow into the cavities.

47. The method of any one of claims 35-46, wherein a surface opposed to the cavity openings is moisture-absorbing.

48. A device, comprising:
an article defining a plurality of cavities each having an opening, at least 5% of the cavities in the article having a cross-sectional dimension of between 10 mm and 14 mm and a depth of between 60 mm and 90 mm,
wherein at least 5% of the cavities contain a superworm.

49. The device of claim 48, wherein at least 50% of the cavities contains only one superworm.

50. The device of any one of claims 48 or 49, wherein at least 90 wt % of the article comprises plastic.

51. The device of any one of claims 48-50, wherein at least 90 wt % of the article comprises wood.

52. The device of any one of claims 48-51, wherein at least 50% of the cavities are cylindrical.

53. The device of any one of claims 48-52, wherein at least 50% of the cavities have substantially identical dimensions.

54. The device of any one of claims 48-53, wherein the article comprises at least 100 cavities.

55. The device of any one of claims 48-54, wherein at least 90% the cavities in the article have a cross-sectional dimension of between 10 mm and 14 mm and a depth of between 60 mm and 90 mm.

56. The device of any one of claims 48-55, wherein a surface opposed to the cavity openings defines a plurality of through-holes.

57. The device of any one of claims 48-56, wherein a surface opposed to the cavity openings permits air flow into the cavities.

58. The device of any one of claims 48-57, wherein a surface opposed to the cavity openings is moisture-absorbing.

59. A device, comprising:
an article defining a plurality of cavities each having an opening, wherein at least 5% of the cavities in the article have a cross-sectional dimension of between 10 mm and 14 mm and a depth of between 60 mm and 90 mm, and wherein a surface opposed to the cavity openings defines a plurality of through-holes.

60. The device of claim 59, wherein at least 50% of the cavities contain a superworm.

61. The device of any one of claims 59 or 60, wherein at least 50% of the cavities contains only one superworm.

62. The device of any one of claims 59-61, wherein at least 90 wt % of the article comprises plastic.

63. The device of any one of claims 59-62, wherein at least 90 wt % of the article comprises wood.

64. The device of any one of claims 59-63, wherein at least 50% of the cavities are cylindrical.

65. The device of any one of claims 59-64, wherein at least 50% of the cavities have substantially identical dimensions.

66. The device of any one of claims 59-65, wherein the article comprises at least 100 cavities.

67. The device of any one of claims 59-66, wherein at least 90% the cavities in the article have a cross-sectional dimension of between 10 mm and 14 mm and a depth of between 60 mm and 90 mm.

68. The device of any one of claims 59-67, wherein the surface opposed to the cavity openings permits air flow into the cavities.

69. The device of any one of claims 59-68, wherein the surface opposed to the cavity openings is moisture-absorbing.

70. A device, comprising:
an article defining a plurality of cavities, wherein at least 5% of the cavities in the article have a cross-sectional dimension of between 10 mm and 14 mm and a depth of between 60 mm and 90 mm, and wherein a surface opposed to the cavity openings has a void area of at least 50%.

71. The device of claim 70, wherein at least 50% of the cavities contain a superworm.

72. The device of any one of claims 70 or 71, wherein at least 50% of the cavities contains only one superworm.

73. The device of any one of claims 70-72, wherein at least 90 wt % of the article comprises plastic.

74. The device of any one of claims 70-73, wherein at least 90 wt % of the article comprises wood.

75. The device of any one of claims 70-74, wherein at least 50% of the cavities are cylindrical.

76. The device of any one of claims 70-75, wherein at least 50% of the cavities have substantially identical dimensions.

77. The device of any one of claims 70-76, wherein the article comprises at least 100 cavities.

78. The device of any one of claims 70-77, wherein at least 90% the cavities in the article have a cross-sectional dimension of between 10 mm and 14 mm and a depth of between 60 mm and 90 mm.

79. The device of any one of claims 70-78, wherein the surface opposed to the cavity openings defines a plurality of through-holes.

80. The device of any one of claims 70-79, wherein the surface opposed to the cavity openings permits air flow into the cavities.

81. The device of any one of claims 70-80, wherein the surface opposed to the cavity openings is moisture-absorbing.

82. A device, comprising:
an article defining a plurality of cavities, wherein at least 5% of the cavities in the article have a cross-sectional dimension of between 10 mm and 14 mm and a depth of between 60 mm and 90 mm, and a surface opposed to the cavity openings permits air flow into the cavity.

83. The device of claim 82, wherein at least 50% of the cavities contain a superworm.

84. The device of any one of claims 82 or 83, wherein at least 50% of the cavities contains only one superworm.

85. The device of any one of claims 82-84, wherein at least 90 wt % of the article comprises plastic.

86. The device of any one of claims 82-85, wherein at least 90 wt % of the article comprises wood.

87. The device of any one of claims 82-86, wherein at least 50% of the cavities are cylindrical.

88. The device of any one of claims 82-87, wherein at least 50% of the cavities have substantially identical dimensions.

89. The device of any one of claims 82-88, wherein the article comprises at least 100 cavities.

90. The device of any one of claims 82-89, wherein at least 90% the cavities in the article have a cross-sectional dimension of between 10 mm and 14 mm and a depth of between 60 mm and 90 mm.

91. The device of any one of claims 82-90, wherein the surface opposed to the cavity openings defines a plurality of through-holes.

92. The device of any one of claims 82-91, wherein the surface opposed to the cavity openings is moisture-absorbing.

93. A device, comprising:
an article defining a plurality of cavities, wherein at least 5% of the cavities in the article have a cross-sectional dimension of between 10 mm and 14 mm and a depth of between 60 mm and 90 mm, and a moisture-absorbing surface opposed to the cavity openings of the plurality of cavities.

94. The device of claim 93, wherein at least 50% of the cavities contain a superworm.

95. The device of any one of claims 93 or 94, wherein at least 50% of the cavities contains only one superworm.

96. The device of any one of claims 93-95, wherein at least 90 wt % of the article comprises plastic.

97. The device of any one of claims 93-96, wherein at least 90 wt % of the article comprises wood.

98. The device of any one of claims 93-97, wherein at least 50% of the cavities are cylindrical.

99. The device of any one of claims 93-98, wherein at least 50% of the cavities have substantially identical dimensions.

100. The device of any one of claims 93-99, wherein the article comprises at least 100 cavities.

101. The device of any one of claims 93-100, wherein at least 90% the cavities in the article have a cross-sectional dimension of between 10 mm and 14 mm and a depth of between 60 mm and 90 mm.

102. The device of any one of claims 93-101, wherein the surface opposed to the cavity openings defines a plurality of through-holes.

103. The device of any one of claims 93-102, wherein the surface opposed to the cavity openings permits air flow into the cavities.

104. A method, comprising:
flowing air through a plurality of cavities defined within an article, wherein at least 5% of the cavities contains a superworm.

105. The method of claim 104, wherein at least 50% of the cavities in the article having a cross-sectional dimension of between 10 mm and 14 mm and a depth of between 60 mm and 90 mm.

106. The method of any one of claims 104 or 105, wherein at least 50% of the cavities contains only one superworm.

107. The method of any one of claims 104-106, wherein at least 90 wt % of the article comprises plastic.

108. The method of any one of claims 104-107, wherein at least 90 wt % of the article comprises wood.

109. The method of any one of claims 104-108, wherein at least 50% of the cavities are cylindrical.

110. The method of any one of claims 104-109, wherein at least 50% of the cavities have substantially identical dimensions.

111. The method of any one of claims 104-110, wherein the article comprises at least 100 cavities.

112. The method of any one of claims 104-111, wherein a surface opposed to the cavity openings defines a plurality of through-holes.

113. The method of any one of claims 104-112, wherein a surface opposed to the cavity openings permits air flow into the cavities.

114. The method of any one of claims 104-113, wherein a surface opposed to the cavity openings is moisture-absorbing.

115. A device, comprising:
an article defining a plurality of cavities each having an opening, at least 5% of the cavities in the article having a cross-sectional dimension of between 10 mm and 14 mm and a depth of between 60 mm and 90 mm.

116. The device of claim 115, wherein at least 50% of the cavities contain a superworm.

117. The device of any one of claims 115 or 116, wherein at least 50% of the cavities contains only one superworm.

118. The device of any one of claims 115-117, wherein at least 90 wt % of the article comprises plastic.

119. The device of any one of claims 115-118, wherein at least 90 wt % of the article comprises wood.

120. The device of any one of claims 115-119, wherein at least 50% of the cavities are cylindrical.

121. The device of any one of claims 115-120, wherein at least 50% of the cavities have substantially identical dimensions.

122. The device of any one of claims 115-121, wherein the article comprises at least 100 cavities.

123. The device of any one of claims 115-122, wherein at least 90% the cavities in the article have a cross-sectional dimension of between 10 mm and 14 mm and a depth of between 60 mm and 90 mm.

124. The device of any one of claims 115-123, wherein a surface opposed to the cavity opening defines a plurality of through-holes.

125. The device of any one of claims 115-124, wherein a surface opposed to the cavity opening permits air flow into the cavities.

126. The device of any one of claims 115-125, wherein a surface opposed to the cavity openings is moisture-absorbing.

127. The device of any one of claims 115-126, wherein a surface opposed to the cavity openings defines a plurality of through-holes.

128. The device of any one of claims 115-127, wherein a surface opposed to the cavity openings permits air flow into the cavities.

129. The device of any one of claims 115-128, wherein a surface opposed to the cavity openings is moisture-absorbing.

130. A method, comprising:
providing an article defining a plurality of cavities; and
using a robot to place superworms within the cavities.

131. A method, comprising:
exposing an article defining a plurality of cavities each having an opening, at least 5% of the cavities in the article having a cross-sectional dimension of between 10 mm and 14 mm and a depth of between 60 mm and 90 mm, to a colony of at least 100 insect larvae.

132. A method, comprising:
exposing an article defining a plurality of cavities each having an opening to a colony of at least 100 insect larvae for a period of time such that the insect larvae move into at least 5% of the cavities in the article.

133. A method, comprising:
exposing an article defining a plurality of cavities each having an opening to a colony of at least 100 insect larvae; and
allowing the insect larvae to search the article for empty cavities.

134. A device, comprising:
an article defining a plurality of cavities each having an opening, at least 5% of the cavities in the article having a cross-sectional dimension of between 10 mm and mm and a depth of between 60 mm and 90 mm,
wherein at least 5% of the cavities contain an insect larvae.

135. A method, comprising:
flowing air through a plurality of cavities defined within an article, wherein at least 5% of the cavities contains an insect larvae.

136. A method, comprising:
providing an article defining a plurality of cavities; and
using a robot to place insect larvae within the cavities.

137. A method, comprising:
exposing an article defining at least 10 cavities each having an opening, a cross-sectional dimension of between 10 mm and 14 mm, and a depth of between 60 mm and 90 mm to a colony of at least 100 superworms.

138. A device, comprising:
an article defining at least 10 cavities each having an opening, a cross-sectional dimension of between 10 mm and 14 mm, and a depth of between 60 mm and 90 mm,
wherein at least 5% of the cavities contain a superworm.

139. A device, comprising:
an article defining at least 10 cavities each having an opening, a cross-sectional dimension of between 10 mm and 14 mm, and a depth of between 60 mm and 90 mm, wherein a surface opposed to the cavity openings defines a plurality of through-holes.

140. A device, comprising:
an article defining at least 10 cavities each having an opening, a cross-sectional dimension of between 10 mm and 14 mm, and a depth of between 60 mm and 90 mm, wherein a surface opposed to the cavity openings has a void area of at least 50%.

141. A device, comprising:
an article defining at least 10 cavities, each having an opening, a cross-sectional dimension of between 10 mm and 14 mm, a depth of between 60 mm and 90 mm, and a surface opposed to the cavity openings that permits air flow into the cavity.

142. A device, comprising:
an article defining at least 10 cavities, each having a cross-sectional dimension of between 10 mm and 14 mm, a depth of between 60 mm and 90 mm, and a moisture-absorbing surface opposed to the cavity openings of the plurality of cavities.

143. A device, comprising:
an article defining at least 10 cavities, each having an opening, a cross-sectional dimension of between 10 mm and 14 mm, and a depth of between 60 mm and 90 mm.

144. A method, comprising:
exposing an article defining at least 10 cavities each having an opening, a cross-sectional dimension of between 10 mm and 14 mm, and a depth of between 60 mm and 90 mm to a colony of at least 100 insect larvae.

145. A device, comprising:
an article defining at least 10 cavities each having an opening, a cross-sectional dimension of between 10 mm and 14 mm, and a depth of between 60 mm and 90 mm,
wherein at least 5% of the cavities contain an insect larvae.
